(19) United States
(12) Patent Application Publication
(10) Pub. No.: US 2014/0132957 A1
(43) Pub. Date: May 15, 2014

(54) OPTICAL MEASUREMENT OF AN ANALYTE

(71) Applicant: Methode Electronics, Inc., Carthage, IL (US)

(72) Inventor: Robert Messerschmidt, Los Altos, CA (US)

(73) Assignee: Methode Electronics, Inc., Carthage, IL (US)

(21) Appl. No.: 13/809,346

(22) PCT Filed: Jul. 11, 2011
(86) PCT No.: PCT/US11/43555

§ 371 Date: Sep. 30, 2013

(51) Int. Cl. G01J 3/42; A61B 5/1455; A61B 5/145; G01N 21/59

(52) U.S. Cl. CPC G01J 3/42; G01N 21/59; A61B 5/1455; A61B 5/14546
USPC 356/409; 356/402; 356/416; 600/314

(57) ABSTRACT

Methods, apparatus and/or systems are disclosed for collecting and processing of spectral data from an object to enable the determination of the presence and/or amount of a target analyte present in the object is disclosed. The object may be a portion of a person's body such as the forearm, palms, fingers, or eye. The target analyte may be ethanol. The method/apparatus/system for performing such spectral data collection may include one or more broadband emitters such as thermal emitters (aka “black-body” or “gray-body” emitters), incandescent sources, light emitting diodes, and the like.

### REFERENCE TO RELATED APPLICATION

The present application claims the benefit of U.S. Provisional Patent Application No. 61/362,922, filed Jul. 9, 2010. Related information is disclosed in U.S. Provisional Patent Application No. 61/362,914, filed Jul. 9, 2010. The disclosures of the above applications are hereby incorporated by reference in their entireties into the present disclosure.

### DESCRIPTION OF RELATED ART

Blood alcohol content (BAC), also called blood alcohol concentration, blood ethanol concentration, or blood alcohol level, is most commonly used as a metric of alcohol intoxication for legal or medical purposes. Blood alcohol tests have a flaw in that they assume that the person being tested is average in various ways.

For example, on average the ratio of blood alcohol content to breath alcohol content (the partition ratio) is 2100 to 1. In other words, there are 2100 parts of alcohol in the blood for every part in the breath. However, the actual ratio in any given individual can vary from 1300:1 to 3100:1, or even more widely. This ratio varies not only from person to person, but within one person from moment to moment. Thus a person with a true blood alcohol level of 0.08 but a partition ratio of 1700:1 at the time of testing would have a 0.10 reading on a Breathalyzer calibrated for the average 2100:1 ratio.

A similar assumption is made in urinalysis. When urine is analyzed for alcohol, the assumption is that there are 1.3 parts of alcohol in the urine for every 1 part in the blood, even though the actual ratio can vary greatly.

Breath alcohol testing further assumes that the test is post-absorptive—that is, that the absorption of alcohol in the subject's body is complete. If the subject is still actively absorbing alcohol, their body has not reached a state of equilibrium where the concentration of alcohol is uniform throughout the body. Most forensic alcohol experts reject test results during this period as the amounts of alcohol in the breath will not accurately reflect a true concentration in the blood.

U.S. Patent Application Publication No. 2006/0002598 teaches a noninvasive alcohol sensor. An illumination subsystem provides light at discrete wavelengths to a skin site of an individual. A detection subsystem receives light scattered from the skin site. A computational unit is interfaced with the detection system. The computational unit has instructions for deriving a spatially distributed multispectral image from the received light at the discrete wavelengths. The computational unit also has instructions for comparing the derived multispectral image with a database of multispectral images to identify the individual.

The illumination subsystem may comprise a light source that provides the light to the plurality of discrete wavelengths and illumination optics to direct the light to the skin site. In some instances, a scanner mechanism may also be provided to scan the light in a specified pattern. The light source may comprise a plurality of quasi-monochromatic light sources, such as LEDs or laser diodes. Alternatively, the light source may comprise a broadband light source, such as an incandescent bulb or glowbar, and a filter disposed to filter light emitted from the broad band source. The filter may comprise a continuously variable filter in one embodiment. In some cases, the detection system may comprise a light detector, an optically dispersive element, and detection optics. The optically dispersive element is disposed to separate wavelength components of the received light, and the detection optics direct the received light to the light detector. In one embodiment, both the illumination and detection subsystems comprise a polarizer. The polarizers may be circular polarizers, linear polarizers, or a combination. In the case of linear polarizers, the polarizers may be substantially crossed relative to each other.

However, it would be desirable to provide a simpler and more compact way of achieving the same result.

### SUMMARY OF THE INVENTION

It is therefore an object of the invention to provide such a simpler and more compact way for optical detection of an analyte such as ethanol.

Methods, apparatus and/or systems are disclosed for collecting and processing of spectral data from an object to enable the determination of the presence and/or amount of a target analyte present in the object is disclosed. The object may be a portion of a person's body such as the forearm, palms, fingers, or eye. The target analyte may be ethanol. The method/apparatus/system for performing such spectral data collection may include one or more broadband emitters such as thermal emitters (aka “black-body” or “gray-body” emitters), incandescent sources, light emitting diodes, and the like. The emitted light may be directed onto one side of an opaque barrier (“blocker”), which is a known optical element that attenuates or limits light passing therethrough, in such a way that light that passes by the barrier may substantially undergo diffuse reflectance within the medium of interest. A detection system may be positioned to receive light from a region of the object of interest on the non-illuminated side of the blocker. This light may interact with one or more optical filters prior to or at the location of the optical detector. The filters may be placed prior to or after the light interacts with the object of interest. The detector may be a single-point detector or a multi-element detector such as a quadrant detector or a 1-D or 2-D detector array. The detector material may be lead-salt, InGaAs, HCT, or other suitable material. One or more optical filters may be selected to measure signals that relate to the analyte of interest and/or materials or optical characteristics of the object of interest. Measurements made of the amount of light passing through the optical filter(s) and incident on the detector(s) may be recorded and further processed to yield an estimate of the presence and/or amount of the analyte present in the object of interest.

In another embodiment, the measurement of the analyte may be made in the space above the object of interest. In particular, transdermal skin measurements may be able to be made at skin sites such as the forehead or palm. A multi-pass optical cell such as a Herriot cell may be incorporated to increase the path length through the vapor. The light source may be a broad-band light source with or without optical filtering, or may be a narrow-band source such as a gas laser, or solid-state laser. A similar method of measurement may be used for analyte vapor emitted from the lachrymal fluid of the eye.

In another embodiment, the diffuse reflectance measurements of the object of interest may be performed with photothermal beam deflection spectroscopy.

### BRIEF DESCRIPTION OF THE DRAWINGS

Preferred embodiments of the present invention will be disclosed in detail with reference to the drawings, in which:

FIG. 1 shows a block diagram of a system implementing any of the preferred embodiments; and

FIG. 2 shows the spectral response of ethanol.

### DETAILED DESCRIPTION OF THE PREFERRED EMBODIMENTS

Preferred embodiments of the present invention will be set forth in detail with reference to the drawings, in which like reference numerals refer to like elements or steps throughout.

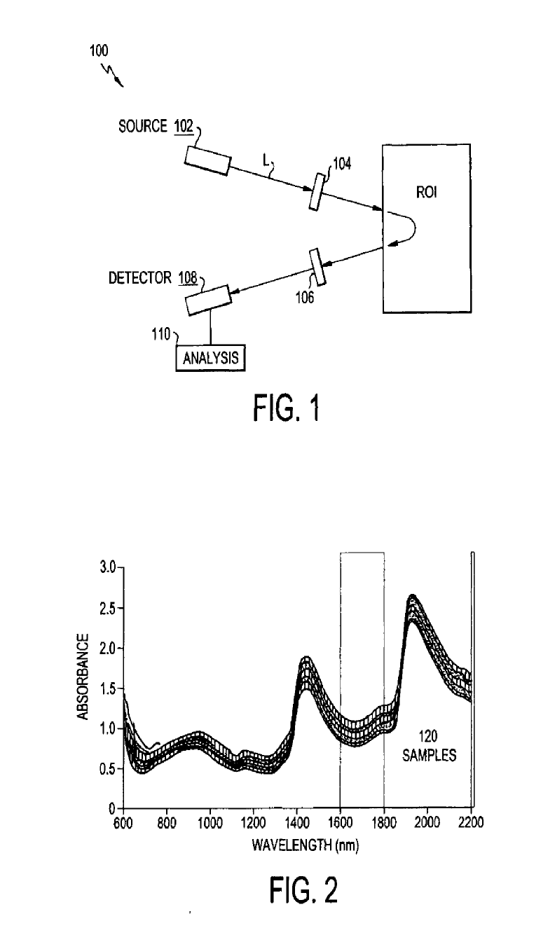

FIG. 1 shows a system 100 for detecting an analyte (such as ethanol) in a region of interest ROI, which can be any suitable part of the human anatomy or vapor given off by human skin or lachrymal fluid. In the system 100, a broadband source 102, such as a thermal emitter (aka “black-body” or “gray-body” emitter), incandescent source, or light emitting diode, emits light L that is directed onto one side of a barrier 104. The light attenuated by the barrier is incident on the region of interest ROI, in which it undergoes diffuse reflectance. The reflected light passes through one or more optical filters 106 and is then incident on a detector 108 to produce a detection signal. The filters 106 can be, e.g., bandpass filters corresponding to the peaks and valleys in the spectroscopic signature of the analyte.

A spectroscopic analysis subsystem 110, which can be any suitably programmed computing device, analyzes the detection signal to detect the peaks and valleys corresponding to the known spectroscopic peaks and valleys of the analyte, e.g., ethanol. FIG. 2 shows those peaks and valleys for ethanol. By detecting and measuring the peaks and valleys, the spectroscopic analysis subsystem can determine both the presence and the concentration of the analyte. In the example of ethanol, the spectroscopic analysis subsystem can determine the presence and concentration of the ethanol and use that information to make an ultimate determination such as blood alcohol content.

While a preferred embodiment has been set forth above, those skilled in the art who have reviewed the present disclosure will readily appreciate that other embodiments can be realized within the scope of the present invention, which should therefore be construed as limited only by the appended claims.

What is claimed is:

1. A method for detecting an analyte in a region of interest, the method comprising:
(a) generating light from a broadband light source;
(b) causing the light to be incident on the region of interest through a barrier to cause diffuse internal reflection of the light within the region of interest;
(c) detecting the light that has been internally reflected within the region of interest, using a detector, to produce a detection signal; and
(d) spectroscopically analyzing the detection signal to detect the analyte.

2. The method of claim 1, wherein the analyte is ethanol.

3. The method of claim 2, wherein the region of interest is a part of the human body.

4. The method of claim 2, wherein the region of interest is a secretion of the human body.

5. The method of claim 4, wherein the secretion is a lachrymal secretion.

6. The method of claim 1, wherein step (c) is performed while at least one optical filter is disposed in a path of the light between the region of interest and the detector.

7. A system for detecting an analyte in a region of interest, the system comprising:
a broadband light source for generating light;
a barrier disposed in a path of the light for causing the light to be incident on the region of interest through a barrier to cause diffuse internal reflection of the light within the region of interest;
a detector disposed to detect the light that has been internally reflected within the region of interest to produce a detection signal; and
an analysis subsystem, receiving the detection signal, for spectroscopically analyzing the detection signal to detect the analyte.

8. The system of claim 7, wherein the analysis subsystem is configured such that the analyte is ethanol.

9. The system of claim 7, further comprising an optical filter disposed to be in the path of the light between the region of interest and the detector.

10. A method for detecting an analyte in a region of interest, the method comprising:
(a) generating light;
(b) causing the light to be incident on the region of interest to cause diffuse internal reflection of the light within the region of interest;
(c) detecting the light that has been internally reflected within the region of interest, using a detector, to produce a detection signal; and
(d) spectroscopically analyzing the detection signal to detect the analyte;
wherein the region of interest is a vapor given off by a living body.

11. The method of claim 10, wherein the analyte is ethanol.

12. The method of claim 11, wherein the vapor is vapor emitted by skin.

13. The method of claim 11, wherein the vapor is vapor emitted by a lachrymal secretion.

14. The method of claim 10, wherein step (c) is performed while at least one optical filter is disposed in a path of the light between the region of interest and the detector.
